(19) United States
(12) Patent Application Publication
(10) Pub. No.: US 2008/0234285 A1
(43) Pub. Date: Sep. 25, 2008

(54) Combination of Organic Compounds

(76) Inventor: David Louis Feldman, Teaneck, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/586,013

(22) PCT Filed: Jan. 21, 2005
(86) PCT No.: PCT/EP2005/000597

§ 371 Date: Jul. 14, 2006

Related U.S. Application Data
(60) Provisional application No. 60/583,222, filed on Jun. 25, 2004.

(51) Int. Cl. A61K 31/496; A61P 35/04; A61P 19/00; A61P 9/00; A61P 17/00; A61P 3/00

(52) U.S. Cl. 514/252.18

(57) ABSTRACT

The invention relates to a combination, such as a combined preparation or pharmaceutical composition, respectively, comprising the renin inhibitor of formula (I) or a pharmaceutically acceptable salt thereof and at least one PDGF receptor tyrosine kinase inhibitor.

The invention relates to a combination, such as a combined preparation or pharmaceutical composition, respectively, comprising a renin inhibitor or a pharmaceutically acceptable salt thereof and at least one PDGF receptor tyrosine kinase inhibitor preferably N-{5-[4-(4-methyl-piperazino-methyl)-benzoylamido]-2-methylphenyl}-4-(3-pyridyl)-2-pyrimidine-amine, or a pharmaceutically acceptable salt thereof.

Thus in a first aspect, the present invention relates a combination, such as a combined preparation or pharmaceutical composition, respectively, comprising as active ingredients;

(i) a renin inhibitor or a pharmaceutically acceptable salt thereof; and

(ii) a least one PDGF receptor tyrosine kinase inhibitor or a pharmaceutically acceptable salt thereof.

The class of renin inhibitors comprises compounds having differing structural features. For example, mention may be made of compounds which are selected from the group consisting of ditekiren (chemical name: [1S-[1R*,2R*,4R*(1R*,2R*)]]-1-[(1,1-dimethylethoxy)carbonyl]-L-proly I-L-phenylalanyl-N-[2-hydroxy-5-methyl-1-(2-methylpropyl)-4-[[[2-methyl-1-[[(2-pyridinylmethyl)amino]carbonyl]butyl]amino]carbonyl]hexyl]-N-alfa-methyl-L-histidinamide); terlakiren (chemical name: [R—(R*,S*)]—N-(4-morpholinylcarbonyl)-L-phenylalanyl-N-[1-(cyclohexylmethyl)-2-hydroxy-3-(1-methylethoxy)-3-oxopropyl]-S-methyl-L-cysteineamide); zankiren (chemical name: [1S-[1R*[R*(R*)],2S*,3R*]]—N[1-(cyclohexylmethyl)-2,3-dihydroxy-5-m ethylhexyl]-alfa-[[2-[[(4-methyl-1-piperazinyl)sulfonyl]methyl]-1-oxo-3-phenylpropyl]amino]-4-thiazolepropanamide), especially the hydrochloride thereof; RO 66-1132 and RO-66-1168 respectively of formula (A) and (B);

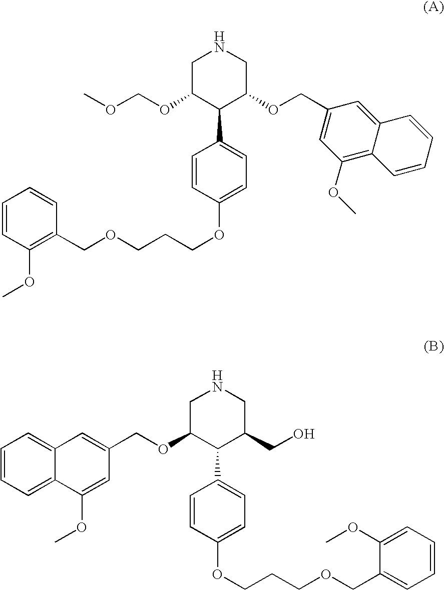

Especially preferred is the compound of formula (I),

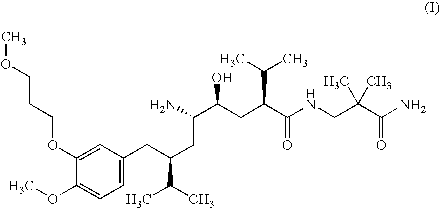

chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide (hereinafter: “aliskiren” [International Non-proprietary Name]), is specifically disclosed in EP 678503 A. Especially preferred is the hemi-fumarate salt thereof.

The term “at least one” shall mean that in addition to the renin inhibitor one or more, for example two, furthermore three, active ingredients as specified according to the present invention can be combined.

The PDGF—R—, tyrosine kinase inhibitors used according to the present invention are preferably selected from the group comprising the following compounds: 4-(4-methylpiperazin-1-ylmethyl)-N-[4-methyl-3-(4-pyridin-3-yl)pyrimidin-2-ylamino)phenyl]-benzamide, 4-Methyl-N-[3-(4-methyl-imidazol-1-yl)-5-trifluoromethyl-phenyl]-3-(4-pyridin-3-yl-pyrimidin-2-ylamino)-benzamide, an inhibitor of PDGF-receptor isoforms, compounds as described in Mahboobi S et al., J. Med. Chem. 2002, 45:1002-1018 and hereby incorporated by reference; the PDGF receptor kinase blocker AG1295 having the CAS Number 71897-07-9; AG1295/96 as described by Kovalenko M et al., Cancer Res. 1994 54:6106-6114 and Ludewig D et al., Cell Tissue Res. 2000, 299:97-103 and hereby incorporated by reference; CT52923 (4-(6,7-dimethoxy-4-quinazolinyl)-N-(3,4-methylenedioxybenzyl)-1-piperazinethiocarboxamide); RP-1776; GFB-111; pyrrolo[3,4-c]-beta-carboline-diones, SU 102 (developed by SUGEN); AG1296 having the CAS Number 146535-11-7; RPR101511A developed by Aventis Pharma; CDP 860 and Zvegf3 developed by ZymoGenetics; CP 673451 and PD 170262 from Pfizer, KI 6783, having the CAS number 190726-45-5, an inhibitor of PDGF—R developed by Kirin Brewery, Japan; KN 1022 developed by Kyowa Hakko in Japan and Millenium Pharmaceuticals in US; AG 13736 developed by Pfizer; CHIR 258 developed by Chiron Corporation; MLN 518 from Millenium Pharmaceuticals and SU 11248 from SUGEN-Pfizer, Leflunomide; or pharmaceutically acceptable salts thereof.

CT52923 has been described by Matsuno K, et al., “Synthesis and structure activity relationships of PDGF receptor phosphorylation inhibitor-1.” in 18th Symposium on Medicinal Chemistry; Nov. 25-27, 1998; Kyoto, Japan, the Pharmaceutical Society of Japan, Division of Medicinal Chemistry, Tokyo, Japan:Abstract 2-P-05.

RP-1776, a cyclic peptide, was isolated from the culture broth of Streptomyces sp. KY11784. It is described, e.g. by Toki S, Agatsuma T, et al., J. Antibiot. (Tokyo) May 2001;54(5):405-14.

GFB-111 is described, e.g. in Blaskovich M A et al., Nat. Biotechnol. October 2000;18(10):1065-70 and in Delarue F. et al, 91^{st} Annual meeting of the American Association for Cancer research, 41:458, 2000.

Pyrrolo[3,4-c]-beta-carboline-diones is described, e.g. by Teller S, Eur. J. Med. Chem. April 2000;35(4):413-27.

CDP 860 is a pegylated antibody fragment derived from the human anti-platelet derived growth factor beta receptor antibody.

PD 170262 or 2-[4-(2-diethlaminoethoxy)phenylamino]-8-methyl-6-(3-thienyl)pyrido[2,3-d]pyrimidin-7(8H)-one is a potent inhibitor of tyrosine kinase with selectivity for the platelet-derived growth factor tyrosine kinase. Synthesis and tyrosine kinase inhibitory activity of a series of 2-amino-8H-pyrido[2,3-d] pyrimidines is described, e.g. in Klutchko S. et al., 213^{th} American Chemical Society National meeting: abst. MEDI 201 (poster), 1997, USA.

KI 6783 or 4-(3,4-dimethoxyphenoxy)-6,7-dimethoxyquinoline is described, e.g. in Kubo K. et al, Bioorganic and Medicinal Chemistry Letters 7:2935-2940, 1997 and Yagi M. et al., Exp. Cell Research 234:285-92, 1997.

KN1022 or 6,7-dimethoxy-4-[4-(4-nitrophenyl)aminocarbonylpiperazin-1yl]-quinazoline, which inhibits PDGFR phosphorylation, is described, e.g. in 217^{th} American Chemical Society National meeting abstr. MEDI 061, Part1, 1999, Japan.

AG 013736 or N-methyl-2-[3-[2-(2-pyridyl)vinyl]-1H-indazole-6-ylsulfanyl]-benzamide is disclosed, e.g. in Heller et al., Pharmacological activities of AG 013736, a small molecule inhibitor of VEGF/PDGFR tyrosine kinases, 93^{rd} Annual meeting f the American association for Cancer research 43:1082, 2002, USA.

CHIR 258 is an orally active amino-benzimidazole quinoline growth factor kinase inhibitor which demonstrated a spectrum of inhibitory activity against receptor tyrosine kinases, e.g. from the PDGFR family. CHIR 258 is disclosed, e.g. in Steigerwalt R et al. and Lee S H et al. in 94^{th} Annual Meeting of the American Association for Cancer Research 753(plus poster) abstr. 3783 and 934 (plus poster) abstr. R4702, respectively, 2003, USA.

SU11248 or 5-[3-fluoro-2-oxo-1,2-dihydroindol-(3Z)-ylidenemethyl]-2,4-dimethyl-1H-pyrrole-3-carboxylic acid(2-diethylaminoethyl)amine is multiple targeted kinase inhibitor with selectivity for, e.g. PDGFR. SU11248 is disclosed, e.g. in Xin L. et al., 93^{rd} Annual Meeting of the American Association for Cancer Research 43:1081 (plus poster), 2002, USA.

MLN 518 is a piperazinyl derivative of quinazoline of formula 4-[4-(N-para-iso-propoxyphenylcarbamoyl)-1-piperazinyl]-6-methoxy-7-(piperidinopropyloxy)-quinazoline that inhibits, e.g. PDGF R phosphorylation in binding assays, it is described, e.g. by Stone R M et al., Blood 102:65-66, 2003, Kelly L M et al., Cancer Cell 1: 421-23, 2002.

Leflunomide (SU 101) or 4-Isoxazolecarboxamide, 5-methyl-N-[4-(trifluoromethyl)phenyl] is a tyrosine kinase inhibitor.

Preferred PDGF receptor tyrosine kinase inhibitors are N-phenyl-2-pyrimidine-amine derivatives of formula II

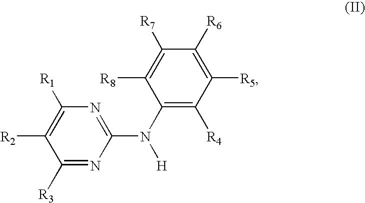

as described in the patent applications EP 0 564 409 A1 and WO 99/03854, incorporated into the present application by reference.

Preference is given above all especially to the corn pound of formula (II) which is CGP 57148B {N-{5-[4-(4-methyl-piperazino-methyl)-benzoylamido]-2-methylphenyl}-4-(3-pyridyl)-2-pyrimidine-amine}. CGP 57148B (hereinafter: “Imatinib” [International Non-proprietary Name]) and the use thereof, especially as an anti-tumour agent, are described in Example 21 of European patent application EP-A-0 564 409, which was published on 6 Oct. 1993, and in equivalent applications and patents in numerous other countries, e.g. in U.S. Pat. No. 5,521,184 and in Japanese patent 2706682. Another preference is given to the β-crystal form of 4-(4-methylpiperazin-1-ylmethyl)-N-[4-methyl-3-(4-pyridin-3-yl)pyrimidin-2-ylamino)phenyl]-benzamide methanesulfonate as described in the European patent application No. 998 473 published on May 10, 2000.

The term “4-(4-methylpiperazin-1-ylmethyl)-N-[4-methyl-3-(4-pyridin-3-yl)pyrimidin-2-yl-amino)phenyl]-benzamide” includes all crystal forms especially the β-crystal form as described in the European patent application No. 998 473.

Very preferably a N-phenyl-2-pyrimidine-amine derivative of formula (II) is used in the form of its monomesylate salt.

The compounds of formula II are generically and specifically disclosed in the patent applications EP 0 564 409 A1 and WO 99/03854, in particular in the compound claims and the final products of the working examples, the subject-matter of the final products, the pharmaceutical preparations and the claims are hereby incorporated into the present application by reference to these publications. Comprised are likewise the corresponding stereoisomers as well as the corresponding polymorphs, e.g. crystal modifications, which are disclosed therein.

In EP 0 564 409 A1 the compounds II are described to be useful for the therapy of cancer, thrombosis, psoriasis, fibrosis, dermatosclerosis and atherosclerosis.

For the purposes of isolation or purification, as well as in the case of compounds that are used further as intermediates, it is also possible to use pharmaceutically unacceptable salts. Only pharmaceutically acceptable, non-toxic salts are used for therapeutic purposes, however, and those salts are therefore preferred.

Further suitable PDGF receptor tyrosine kinase inhibitors are disclosed in WO 98/35958, especially the compound of Example 62, and U.S. Pat. No. 5,093,330 in each case in particular in the compound claims and the final products of the working examples, the subject-matter of which are hereby incorporated into the present application by reference to these publications.

Other preferred compounds are described in the patent application WO 04/005281, especially the examples, most preferably the compound of example 92 of formula

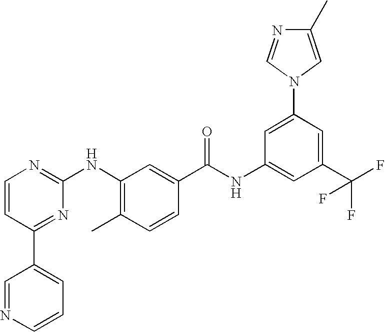

which is also known as 4-Methyl-N-[3-(4-methyl-imidazol-1-yl)-5-trifluoromethyl-phenyl]-3-(4-pyridin-3-yl-pyrimidin-2-ylamino)-benzamide.

Preferred PDGF receptor tyrosine kinase inhibitors are selected from 4-(4-methylpiperazin-1-ylmethyl)-N-[4-methyl-3-(4-pyridin-3-yl)pyrimidin-2-ylamino)phenyl]-benzamide (imatinib), 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, CT52923 (4-(6,7-dimethoxy-4-quinazolinyl)-N-(3,4-methylenedioxybenzyl)-1-piperazinethiocarboxamide), RP-1776, GFB-111, pyrrolo[3,4-c]-beta-carboline-diones, SU 102 (developed by SUGEN), AG1296 (CAS Number 146535-11-7), AG1296 (CAS Number 71897-07-9) and RPR101511A or, in each case, a pharmaceutically acceptable salt thereof.

In each case where appropriate, e.g. if the compound is not present as a pharmaceutically acceptable salt per se as in the case of hydrochlorothiazide, these compounds also include their pharmaceutically acceptable salts.

The corresponding active ingredients or a pharmaceutically acceptable salts thereof may also be used in form of a solvate, such as a hydrate or including other solvents, used for crystallization.

The most preferred PDGF receptor tyrosine kinase inhibitors are N-{5-[4-(4-methyl-piperazino-methyl)benzoylamido]-2-methylphenyl}-4-(3-pyridyl)-2-pyrimidine-amine (imatinib) and 4-Methyl-N-[3-(4-methyl-imidazol-1-yl)-5-trifluoromethyl-phenyl]-3-(4-pyridin-3-yl-pyrimidin-2-ylamino)-benzamide or in each case a pharmaceutically acceptable salt thereof such as the mono-hydrochloride.

Preferred are combinations, such as combined preparations or pharmaceutical compositions, respectively, comprising a DPP-IV inhibitor preferably LAF237 or a pharmaceutically accepted salt thereof and as second active agent an active agent selected from the group consisting of 4-(4-methylpiperazin-1-ylmethyl)-N-[4-methyl-3-(4-pyridin-3-yl)pyrimidin-2-ylamino)phenyl]-benzamide (imatinib), 4-(4-methylpiperazin-1-ylmethyl)-N-[4-methyl-3-(4-pyridin-3-yl)pyrimidin-2-ylamino)phenyl]-benzamide methanesulfonate, CT52923 (4-(6,7-dimethoxy-4-quinazolinyl)-N-(3,4-methylenedioxybenzyl)-1-piperazinethiocarboxamide), 4-Methyl-N-[3-(4-methyl-imidazol-1-yl)-5-trifluoromethyl-phenyl]-3-(4-pyridin-3-yl-pyrimidin-2-ylamino)-benzamide, RP-1776, GFB-111, pyrrolo[3,4-c]-beta-carboline-diones, SU 102 (developed by SUGEN), AG1296 (CAS Number 146535-11-7), AG1296 (CAS Number 71897-07-9) and RPR101511A, or in each case, a pharmaceutically acceptable salt thereof.

The corresponding active ingredients or a pharmaceutically acceptable salt thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

All of these marketed products may be utilized in as such for combination therapy according to the present invention.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. The subject matter of the above mentioned references especially the specifically described compounds e.g. in the claims or examples, are herewith incorporated by reference in this specification.

Any person skilled in the art is fully enabled to identify the active agents and, based on these references, likewise enabled to manufacture and test the pharmaceutical indications and properties in standard test models, both in vitro and in vivo.

A preferred PDGF receptor tyrosine kinase inhibitor is selected from N-{5-[4-(4-methyl-piperazino-methyl)-benzoylamido]-2-methylphenyl}-4-(3-pyridyl)-2-pyrimidine-amine (imatinib) and 4-Methyl-N-[3-(4-methyl-imidazol-1-yl)-5-trifluoromethyl-phenyl]-3-(4-pyridin-3-yl-pyrimidin-2-ylamino)-benzamide, or in each case a pharmaceutically acceptable salt thereof such as the mono-hydrochloride.

A preferred renin inhibitor is 2(S),4(S),5(S),7(S)—N-(3-amino-2,2-dimethyl-3-oxopropyl)-2,7-di(1-methylethyl)-4-hydroxy-5amino-8-[4-methoxy-3-(3-methoxy-propoxy)phenyl]-octanamide (aliskiren) or a pharmaceutically acceptable salt thereof such as a hemi-fumarate salt thereof.

Thus the present invention preferably relates to a combination, such as a combined preparation or pharmaceutical composition, respectively, comprising as active ingredients;

(i) 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide or a pharmaceutically acceptable salt thereof; and

(ii) a PDGF receptor tyrosine kinase inhibitor selected from N-{5-[4-(4-methyl-piperazino-methyl)-benzoylamido]-2-methylphenyl}-4-(3-pyridyl)-2-pyrimidine-amine and 4-Methyl-N-[3-(4-methyl-imidazol-1-yl)-5-trifluoromethyl-phenyl]-3-(4-pyridin-3-yl-pyrimidin-2-ylamino)-benzamide, or in each case a pharmaceutically acceptable salt thereof.

The corresponding active ingredients or a pharmaceutically acceptable salts thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

The pharmaceutical activities as effected by administration of the renin inhibitor especially aliskiren of formula (I) or of the combination of the active agents used according to the present invention can be demonstrated e.g. by using corresponding pharmacological models known in the pertinent art. The person skilled in the pertinent art is fully enabled to select a relevant animal test model to prove the hereinbefore and hereinafter indicated therapeutic indications and beneficial effects.

To evaluate the antihypertensive activity of the combination according to the invention, for example, the methodology as described by Lovenberg W: Animal models for hypertension research. Prog. Clin. Biol. Res. 1987, 229, 225-240 may be applied.

For the evaluation that the combination according to the present invention may be used for the treatment of congestive heart failure, for example, the methods as disclosed by Smith H J, Nuttall A: Experimental models of heart failure. Cardiovasc Res 1985, 19, 181-186 may be applied. Molecular approaches such as transgenic methods are also described, for example by Luft et al.: Hypertension-induced end-organ damage. “A new transgenic approach for an old problem.” Hypertension 1999, 33, 212-218.

The evaluation of the cardiovascular benefic effects especially in diabetes of the agents given alone or in combination can be performed using models such as the Zucker fatty rat as described in the publication of Nawano et al., Metabolism 48: 1248-1255, 1999. Also, studies using diabetic spontaneously hypertensive rats are described in the publication of Sato et al., Metabolism 45:457-462, 1996.

The corresponding subject matter of these references is herewith incorporated by reference in this specification.

Combinations of the invention can also be determined by other test models known as such to the person skilled in the pertinent art or by clinical trials.

The person skilled in the pertinent art is fully enabled to select a relevant test model to prove the herein indicated therapeutic indications and beneficial effects (i.e. good therapeutic margin, improved therapeutic efficacy, no action on hypertension, and other advantages). The pharmacological activity may, for example, be demonstrated in a clinical study or in the test procedure as essentially described hereinafter in a manner known to the skilled person.

Accordingly, the combination according to the present invention may be used, e.g., for the prevention, delay of progression or treatment of diseases and disorders that may be inhibited by the renin inhibitors, especially of formula (I), or that may be inhibited by PDGF receptor tyrosine kinase inhibitors.

Especially, the combination according to the present invention may be used, e.g., for the prevention, delay of progression or treatment of diseases and disorders selected from the group consisting of cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, Nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance.

Cardiac, vascular or kidney hypertrophy or hypertrophic remodeling is characterized by an increase in mass of the heart, arteries, large vessels or kidney.

The combination of the invention is particularly useful for treating and/or preventing injuries in relation to hypertension. Hypertension, a condition of elevated blood pressure, affects a substantial number of the human population. Consequences of persistent hypertension include vascular damage to the ocular, renal, cardiac and cerebral systems, and the risk of these complications increases as blood pressure increases. Basic factors controlling blood pressure are cardiac output and peripheral vascular resistance, with the latter being the predominant common mechanism which is controlled by various influences. Injuries in relation to hypertension, according to the invention are preferably but not limited to heart failure, cardiac hypertrophy such as right or left ventricular hypertrophy (LVH), renal arteriopathy, and vascular diseases e.g. hypertrophic medial thickening in arteries and/or in large vessels, mesenteric vasculature hypertrophy, restenosis or atherosclerosis.

Preferably, said combination may be used for the treatment of hypertension, especially ISH, congestive heart failure, endothelial dysfunction, impaired vascular compliance, vascular restenosis,

Preferably, said combination may be used for the treatment of hypertension-induced cardiovascular diseases or hypertension-induced vascular diseases.

A “disease or condition which may be inhibited by the renin inhibitor of formula (I)” as defined in this application comprises, but is not limited to hypertension, congestive heart failure, diabetes, especially type 2 diabetes mellitus, diabetic retinopathy, macular degeneration, diabetic nephropathy, glomerulosclerosis, renal failure, especially chronic renal failure, diabetic neuropathy, syndrome X, premenstrual syndrome, coronary heart disease, angina pectoris, myocardial infarction, stroke, vascular restenosis, endothelial dysfunction and the like.

Hypertension, in connection with injuries in relation to hypertension, includes and is not limited to mild, moderate and severe hypertension as defined in Journal of Hypertension 1999, 17:151-183, especially on page 162. Especially preferred is “isolated systolic hypertension” (ISH).

Preferably, the jointly therapeutically effective amounts of the active agents according to the combination of the present invention can be administered simultaneously or sequentially in any order, e.g. separately or in a fixed combination.

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of the renin inhibitor preferably aliskiren and at least one PDGF receptor tyrosine kinase inhibitor preferably imatinib, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with hypertension, e.g. less cardiovascular side effects. An additional and preferred aspect of the present invention is the prevention, delay of progression or treatment of the condition of isolated systolic hypertension and impaired vascular compliance which means decreased vascular elasticity.

The term “potentiation” shall mean an increase of a corresponding pharmacological activity or therapeutical effect, respectively. Potentiation of one component of the combination according to the present invention by co-administration of another component according to the present invention means that an effect is being achieved that is greater than that achieved with one component alone.

The term “synergistic” shall mean that the drugs, when taken together, produce a total joint effect that is greater than the sum of the effects of each drug when taken alone.

ISH is the most common form of hypertension in people over 50 years. It is defined as elevated systolic blood pressure (above 140 mm Hg) in conjunction with normal diastolic blood pressure (below 90 mm Hg). Elevated systolic blood pressure is an independent risk factor for cardiovascular diseases and may lead e.g. to myocardial hypertrophy and heart failure. ISH is furthermore characterized by an increased pulse pressure, defined as the difference between systolic and diastolic blood pressures. Elevated pulse pressure is being recognized as the type of hypertension the least likely to be well controlled. A reduction of elevated systolic blood pressure and correspondingly of pulse pressure is associated with a significant risk reduction in cardiovascular death. It has surprisingly been found that the combination of renin inhibitor of formula (I) and a PDGF receptor tyrosine kinase inhibitor leads to a decrease of ISH and pulse rate, both in hypertensive patients having type 2 diabetes mellitus and in hypertensive patients that do not have type 2 diabetes mellitus.

Furthermore, it has been found that the chronic co-administration of a PDGF receptor tyrosine kinase inhibitor imparts the beneficial effect on blood vessel morphology and function and results in a decrease of vascular stiffness and correspondingly in a maintenance and in an improvement of vascular compliance. It has also been found that the chronic co-administration of a PDGF receptor tyrosine kinase inhibitor and a renin inhibitor imparts the beneficial effect on cardiac morphology and function.

Accordingly, it has been found that the addition of a PDGF receptor tyrosine kinase inhibitor to that of renin inhibitors preferably of formula (I) would potentiate the effect on systolic blood pressure and further improve vascular stiffness/compliance and also reduce cardiovascular side effects. Conversely, the proven antihypertensive effects of the renin inhibitors on systolic and diastolic blood pressure may be potentiated by the addition of a PDGF receptor tyrosine kinase inhibitor. The benefit of these combinations may also extend to an additional or potentiated effect on endothelial function, and improve vascular function and structure in various organs/tissues including the kidney, heart, eye and brain. Through the use of this combination, an anti-thrombotic and anti-atherosclerotic effect can also be demonstrated. This effect proves to be highly beneficial by evoking an additive or synergistic effect on cardiovascular function/structure when administered with the renin inhibitor of formula (I) which alone improves cardiovascular function and structure through a distinct mechanism.

Combined administration of a renin inhibitor with a PDGF receptor tyrosine kinase inhibitor will evoke further antihypertensive effects, improve vascular dynamics in hypertensive patients to a greater extent than after administration of either agent given alone.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

For example, it has turned out that the combination according to the present invention provides benefit especially in the treatment of modest hypertension or isolated systolic hypertension that is beneficial to all diabetic patients regardless of their hypertensive status, e.g. reducing the risk of negative cardiovascular events by two different modes of action.

The renin inhibitors especially of formula (I) have proven to be also useful in the treatment of type 2 diabetes mellitus beyond the reduction of blood pressure in for example improving microalbuminuria. At sub-therapeutic doses, with respect to the treatment of hypertension, the combination according to the invention may be merely used for the treatment of diabetes, especially type 2 diabetes mellitus. In view of the reduced dose of the renin inhibitor of formula (I), there is a considerable safety profile of the combination making it suitable for improved therapy.

Thus the present invention furthermore concerns;

1) A combination according to the present invention for use as a medicament.

2) The use of a renin inhibitor preferably of formula (I) or a pharmaceutically acceptable salt thereof in combination with a least one PDGF receptor tyrosine kinase inhibitor or a pharmaceutically acceptable salt thereof,

for the manufacture of a medicament for the prevention, delay of progression or treatment of a disease and disorder selected from selected from cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, Nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance.

3) A method for the prevention, delay of progression or treatment of a disease and disorder selected from selected from cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, Nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance, comprising administering to a warm-blooded animal, including man, in need thereof jointly therapeutically effective amounts of

(i) a renin inhibitor preferably of formula (I) or a pharmaceutically acceptable salt thereof;

(ii) a least one PDGF receptor tyrosine kinase inhibitor or a pharmaceutically acceptable salt thereof.

4) A pharmaceutical composition for the prevention of, delay of progression of, treatment of a disease or condition selected from the group consisting of cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, Nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance;

comprising as active ingredients

(i) a renin inhibitor preferably of formula (I) or a pharmaceutically acceptable salt thereof;

(ii) a least one PDGF receptor tyrosine kinase inhibitor or a pharmaceutically acceptable salt thereof;

and at least one additional pharmaceutically acceptable carrier.

Method or use as described above, wherein the renin inhibitor is administered simultaneously with the PDGF receptor tyrosine kinase inhibitor or sequential in time with the PDGF receptor tyrosine kinase inhibitor.

Method or use as described above, wherein the renin inhibitor and the PDGF receptor tyrosine kinase inhibitor are administered in the form of a combination of the present invention such as a fixed combination or combined preparation or kit of part.

Method or use as described above, for treating and/or preventing injuries in relation to hypertension.

Method or use as described above, for treating and/or preventing injuries in relation to hypertension wherein the patient is suffering from hypertension or in hypertensive patients having type 2 diabetes mellitus.

Method or use as described above, for treating and/or preventing heart failure, cardiac hypertrophy such as right or left ventricular hypertrophy (LVH), renal arteriopathy, and vascular diseases e.g. hypertrophic medial thickening in arteries and/or in large vessels, mesenteric vasculature hypertrophy, restenosis or atherosclerosis wherein the patient is suffering from diabetes preferably type 2 diabetes mellitus.

The pharmaceutical compositions according to the present invention as described hereinbefore and hereinafter may be used for simultaneous use or sequential use in any order, for separate use or as a fixed combination.

Preferred are combinations, such as a combined preparations or pharmaceutical compositions, respectively, comprising the renin inhibitor of formula (I) or a pharmaceutically accepted salt thereof and as second active agent an active agent selected from the group consisting of imatinib, CT52923, RP-1776, GFB-111, pyrrolo[3,4-c]-beta-carboline-diones, SU 102, AG1296, AG1296 and RPR101511A.

The pharmaceutical composition according to the present invention comprises a “kit of parts” in the sense that the components can be dosed independently or by use of different fixed combinations with distinguished amounts of the components at different time points. The parts of the “kit of parts” can then e.g. be administered simultaneously or chronologically staggered, that is at different time points and with equal or different time intervals for any part of the “kit of parts”. Preferably, the time intervals are chosen such that the effect on the treated disease or condition in the combined use of the parts is larger than the effect that would be obtained by use of only any one of the components. Preferably, there is at least one beneficial effect, e.g. a mutual enhancing of the effect of

(i) a renin inhibitor preferably of formula (I) or a pharmaceutically acceptable salt thereof;

(ii) a least one PDGF receptor tyrosine kinase inhibitor or a pharmaceutically acceptable salt thereof;

in particular a potentiation or a synergism, e.g. a more than additive effect, additional advantageous effects, less side effects, a combined therapeutical effect in a non-effective dosage of one or each of the components, especially a potentiation or a strong synergism.

The invention furthermore relates to a commercial package comprising the combination according to the present invention together with instructions for simultaneous, separate or sequential use.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubilizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

The renin inhibitor of formula (I) will be supplied in the form of suitable dosage unit form, for example, a capsule or tablet, and comprising a therapeutically effective amount, e.g. from about 10 to about 500 mg, of the renin inhibitor of formula (I) which may be applied to patients. Corresponding doses may be taken, for example, in the morning, at mid-day or in the evening. Preferred is b.i.d. administration.

N-{5-[4-(4-methyl-piperazino-methyl)-benzoylamido]-2-methylphenyl}-4-(3-pyridyl)-2-pyrimidine-amine monomesylate, is preferably administered to a human in a dosage in the range of about 2.5 to 850 mg/day, more preferably 5 to 600 mg/day and most preferably 20 to 300 mg/day. Unless stated otherwise herein, the compound is preferably administered from one to four times per day.

### GALENIC FORMULATION—EXAMPLE 1

Film-Coated Tablets

The following constituents are processed for the preparation of 10 000 tablets each containing 100 mg of active ingredient:

| | |
|---|---|
| hemi-fumarate of the compound of formula (I) | 1000 g |
| corn starch | 680 g |
| colloidal silicic acid | 200 g |
| magnesium stearate | 20 g |
| stearic acid | 50 g |
| sodium carboxymethyl starch | 250 g |
| water | quantum satis |

A mixture of one of the compounds of formula I mentioned in the preceding Examples as active ingredient, 50 g of corn starch and the colloidal silicic acid is processed into a moist mass with starch paste prepared from 250 g of corn starch and 2.2 kg of demineralised water. The mass is forced through a sieve having a mesh size of 3 mm and dried at 45° for 30 minutes in a fluidised bed drier. The dried granules are pressed through a sieve having a mesh size of 1 mm, mixed with a previously sieved mixture (1 mm sieve) of 330 g of corn starch, the magnesium stearate, the stearic acid and the sodium carboxymethyl starch, and compressed to form slightly biconvex tablets.

### GALENIC FORMULATION—EXAMPLE 2

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate (optionally in its β-crystal form)

Capsules containing 119.5 mg of the compound named in the title (=COMPOUND I mesylate) corresponding to 100 mg of COMPOUND I (free base) as active substance are prepared in the following composition:

Composition

| | | |
|---|---|---|
| COMPOUND I mesylate | 119.5 | mg |
| Cellulose MK GR | 92 | mg |
| Crospovidone XL | 15 | mg |
| Aerosil 200 | 2 | mg |
| Magnesium stearate | 1.5 | mg |
| | 230 | mg |

The capsules are prepared by mixing the components and filling the mixture into hard gelatin capsules, size 1.

These examples illustrate the invention without in any way limiting its scope.

1. A pharmaceutical composition comprising;
(i) a renin inhibitor or a pharmaceutically acceptable salt thereof; and
(ii) a least one PDGF receptor tyrosine kinase inhibitor or a pharmaceutically acceptable salt thereof.

2. The composition of claim 1 wherein the PDGF receptor tyrosine kinase inhibitors are selected from 4-(4-methylpiperazin-1-ylmethyl)-N-[4-methyl-3-(4-pyridin-3-yl)pyrimidin-2-ylamino)phenyl]-benzamide, 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, CT52923, (4-(6,7-dimethoxy-4-quinazolinyl)-N-(3,4-methylenedioxybenzyl)-1-piperazinethiocarboxamide), RP-1776, GFB-111, pyrrolo[3,4-c]-beta-carboline-diones, SU 102, AG1296, AG1296 and RPR101511A or, in each case, a pharmaceutically acceptable salt thereof.

3. The composition of claim 1 wherein the renin inhibitor is selected from 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, detikiren, terlakiren and zankiren, or a pharmaceutically acceptable salt thereof.

4. The composition of claim 1 wherein the renin inhibitor is 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, or a pharmaceutically acceptable salt thereof.

5. A pharmaceutical composition comprising;
(i) 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide or a pharmaceutically acceptable salt thereof; and
(ii) a PDGF receptor tyrosine kinase inhibitor selected from N-{5-[4-(4-methyl-piperazino-methyl)-benzoylamido]-2-methylphenyl}-4-(3-pyridyl)-2-pyrimidine-amine and 4-Methyl-N-[3-(4-methyl-imidazol-1-yl)-5-trifluoromethyl-phenyl]-3-(4-pyridin-3-yl-pyrimidin-2-ylamino)-benzamide, or in each case a pharmaceutically acceptable salt thereof.

6. The composition of claim 4 wherein the active ingredient (i) is in the form of its hemi-fumarate salt, and the active ingredient (ii) is in the form of a its monomesylate salt.

7. (canceled)

8. A method for the prevention, delay of progression or treatment of a disease and disorder selected from cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance, comprising administering to a warm-blooded animal in need thereof a therapeutically effective amount of a pharmaceutical composition of claim 1.

9. (canceled)

10. A kit of parts comprising
(i) an amount of a renin inhibitor in a first unit dosage form;
(i) an amount of at least one PDGF receptor tyrosine kinase inhibitor, or,
in each case, where appropriate, a pharmaceutically acceptable salt thereof, in the form of two or three or more separate units of the components (i) to (ii).

11. A method of using a kit of parts according to claim 10, wherein the renin inhibitor is selected from the group consisting of aliskiren, detikiren, terlakiren, and zankiren.

12. A method of using the kit of parts according to claim 10, wherein the PDGF receptor tyrosine kinase inhibitors are selected from 4-(4-methylpiperazin-1-ylmethyl)-N-[4-methyl-3-(4-pyridin-3-yl)pyrimidin-2-ylamino)phenyl]-benzamide, 4-(4-methylpiperazin-1-ylmethyl)-N-[4-methyl-3-(4-pyridin-3-yl)pyrimidin-2-ylamino)phenyl]-benzamide methanesulfonate, 4-Methyl-N-[3-(4-methyl-imidazol-1-yl)-5-trifluoromethyl-phenyl]-3-(4-pyridin-3-yl-pyrimidin-2-ylamino)-benzamide, CT52923, (4-(6,7-dimethoxy-4-quinazolinyl)-N-(3,4-methylenedioxybenzyl)-1-piperazinethiocarboxamide), RP-1776, GFB-111, pyrrolo[3,4-c]-beta-carboline-diones, SU 102, AG1296, AG1296 and RPR101511A or, in each case, a pharmaceutically acceptable salt thereof.

13. (canceled)

14. (canceled)

15. The pharmaceutical composition according to claim 2 wherein the renin inhibitor is selected from 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, detikiren, terlakiren and zankiren, or a pharmaceutically acceptable salt thereof.

16. The pharmaceutical composition of claim 5 wherein the active ingredient (i) is in the form of its hemi-fumarate salt, and the active ingredient (ii) is in the form of a its monomesylate salt.

17. A method for the prevention, delay of progression or treatment of a disease and disorder selected from cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance, comprising administering to a warm-blooded animal in need thereof therapeutically effective amount of a pharmaceutical compositor according to claim 2.

18. A method for the prevention, delay of progression or treatment of a disease and disorder selected from cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance, comprising administering to a warm-blooded animal in need thereof therapeutically effective amount of a pharmaceutical compositor according to claim 3.

19. A method for the prevention, delay of progression or treatment of a disease and disorder selected from cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance, comprising administering to a warm-blooded animal in need thereof therapeutically effective amount of a pharmaceutical compositor according to claim 4.

20. A method for the prevention, delay of progression or treatment of a disease and disorder selected from cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance, comprising administering to a warm-blooded animal in need thereof therapeutically effective amount of a pharmaceutical compositor according to claim 5.

21. A method for the prevention, delay of progression or treatment of a disease and disorder selected from cancer, thrombosis, psoriasis, fibrosis, dermatosclerosis, atherosclerosis, restenosis, cardiovascular hypertrophy or cardiovascular hypertrophic remodeling or hypertension-induced cardiovascular diseases, cardiac hypertrophy, cardiac remodeling after myocardial infarction, pulmonary congestion and cardiac fibrosis in dilated or in hypertrophic cardiomyopathy, left or right ventricular hypertrophy, diabetic myopathy, stroke prevention in congestive heart failure, hypertrophic medial thickening in arteries and/or in large vessels, hypertension-induced vascular injuries, mesenteric vasculature hypertrophy, renal hyperfiltration such as after portal renal ablation, proteinuria in chronic renal disease, renal arteriopathy as a consequence of hypertension, nephrosclerosis or hypertensive nephrosclerosis, mesanglial hypertrophy, hypertension, congestive heart failure, diabetes, especially type 2 diabetes mellitus, diabetic retinopathy, macular degeneration, diabetic nephropathy, glomerulosclerosis, chronic renal failure, diabetic neuropathy, syndrome X, premenstrual syndrome, coronary heart disease, angina pectoris, myocardial infarction, stroke, vascular restenosis, macular degeneration, cataracts, premenstrual syndrome, skin and connective tissue disorders, endothelial dysfunction and impaired vascular compliance, comprising administering to a warm-blooded animal in need thereof therapeutically effective amount of a pharmaceutical compositor according to claim 6.
